(19) United States
(12) Patent Application Publication
(10) Pub. No.: US 2012/0065480 A1
(43) Pub. Date: Mar. 15, 2012

(54) STRESS MONITOR SYSTEM AND METHOD

(76) Inventors: Fabio F. Badilini, Brescia (IT); Daniela Lucini, Milano (IT); Massimo Pagani, Milano (IT); Alberto Porta, Mantova (IT)

(21) Appl. No.: 13/257,152

(22) PCT Filed: Mar. 16, 2010
(86) PCT No.: PCT/US10/27472

§ 371 Date: Nov. 28, 2011

Related U.S. Application Data
(60) Provisional application No. 61/161,092, filed on Mar. 18, 2009.

(51) Int. Cl. A61B 5/00

(52) U.S. Cl. 600/301

(57) ABSTRACT

A stress monitoring method includes the steps of acquiring a plurality of individual readings of at least one physiologic data parameter over a period of time, storing the plurality of individual readings, determining the average of at least a portion of the plurality of individual readings, and comparing at least one individual reading to the average to identify any differences between the average and the at least one individual reading.

### CROSS-REFERENCE TO RELATED APPLICATIONS

This application claims the benefit of co-pending U.S. Provisional Patent Application No. 61/161,092, filed on Mar. 18, 2009, which is fully incorporated herein by reference.

### TECHNICAL FIELD

The present invention relates to health monitoring systems and, more particularly, to a system and method for monitoring stress by acquiring, processing, and displaying physiological data.

### BACKGROUND INFORMATION

Stress is generally considered to represent the body's physiologic, biochemical, or neuroendocrine response to, or the pathologic result of interaction with, an external stimulus or challenge commonly referred to as a stressor. When faced with a stressor, such as a threat to one's physical safety or emotional equilibrium, the body responds by exhibiting what is commonly known as a “flight or fight” response. When one experiences the “flight or fight” response, one's heart beats faster, blood pressure rises, and other body systems prepare to meet the perceived threat. This adaptive response generally includes the brain's activation of the autonomic nervous system (ANS), an involuntary system of nerves which controls and stimulates, among other things, the output of two hormones including cortisol from the adrenal cortex and adrenalin from the adrenal medulla. Each of these hormones helps one cope with stress by keeping one alert by increasing heart rate and blood pressure and quickly mobilizing energy reserves, in the case of adrenalin, and by replenishing energy supplies and readying one's immune system to handle bacterial and viral threats, in the case of cortisol.

When exposure to a stressor disrupts the body's homeostasis, the body can either regain its normal equilibrium once the stress has passed, become stuck in an over-aroused state, or become stuck in an under-aroused state. However, the more the body's stress response is activated, the more difficulty the body has returning to an equilibrium state. Instead of leveling off once the stressor has passed, one's stress hormones, heart rate, and blood pressure tend to remain elevated the more frequently one experiences stress. Extended or repeated activation of the stress response takes a heavy toll on the body. Although humans are physiologically equipped to respond to acute stressors, chronic stress results in harmful effects on human health. While the ANS provides protection from acute stressors by speeding up the body during emergencies, the hyperactivity of the ANS can adversely impact one's health by increasing or decreasing hormone production which, if prolonged, can have harmful effects on the body's metabolism, cardiovascular system, and immune system.

The body's metabolism is adversely affected by increased cortisol secretion which produces elevated levels of insulin which can lead to the onset of type 2 diabetes. Chronic increased cortisol secretion has also been shown to lead to gradual demineralization of bone, hypertension, obesity, and cognitive impairment.

The cardiovascular system is also harmed by hyperactivity of the ANS due to increased blood pressure, including blood pressure surges, which can accelerate hardening of the arteries and lead to arteriosclerosis. Chronic increases in cardiovascular activity has also been shown to lead to heart disease, increased risk of heart attack, stroke, kidney disease, and angina due at least in part to increased blood clotting and elevated levels of blood cholesterol.

Although acute stress actually helps the immune system handle a pathogen, chronic stress impairs the ability of the immune system to relocate immune cells to tissue where they are needed to do their job of responding to the pathogenic agent. This immune system suppression compromises one's ability to fight off disease and infection as well as one's capacity to remember or store information by impairing excitability and promoting atrophy of nerve cells in the hippocampus portion of the brain.

The detrimental effects of chronic stress have also been shown to lead to at least four categories of symptoms including physical, cognitive, emotional, and behavioral. Physical symptoms of chronic stress include chronic pain, muscle tension and stiffness, diarrhea or constipation, nausea, dizziness, insomnia, chest pain, rapid heartbeat, weight gain or loss, skin breakouts, loss of sex drive, frequent colds, infertility, migraines, ulcers, heartburn, and high blood pressure. Cognitive symptoms include memory problems, indecisiveness, inability to concentrate, trouble thinking clearly, poor judgment, anxiousness, chronic worrying, loss of objectivity, and fearful anticipation. Emotional symptoms of chronic stress include moodiness, agitation, restlessness, short temper, irritability, impatience, feeling overwhelmed, sense of loneliness and isolation, and depression. Behavioral symptoms generally include eating disorders, sleeping too much or too little, seeking isolation from others, procrastination, neglecting responsibilities, substance abuse, nervous habits, teeth grinding or jaw clenching, and overreacting to unexpected problems. The specific symptoms of stress vary widely from person to person. Some people primarily experience physical symptoms while in others, the stress pattern centers around emotional symptoms and for still others, changes in the way they think or behave predominate.

Because of the widespread damage chronic stress can cause, it's essential to learn techniques to deal with chronic stress in a more positive way in order to reduce its impact on one's daily life. In order to deal with chronic stress, many treatment options have been developed often depending on the specific disorder and the nature of its effect on a specific person. In some cases, treatment is limited to relieving the particular physical symptom involved. However, often the symptoms of stress are cognitive or emotional requiring psychological treatments directed at helping the individual relieve the source of stress or else to learn to cope more effectively with it. Still other symptoms are a combination of one or more category of symptoms requiring a combination of physical and psychological treatments. Some examples of treatments include pharmacologic treatments such as sedatives, tranquilizers, antidepressants, and beta blockers. Other approaches for dealing with stress are behavioral such as physical exercise, recreation, hobbies, involvement in social organizations, and religious activities. Relaxation techniques such as meditation, guided imagery, progressive muscular relaxation, and hypnosis have also been recommended as effective ways to deal with stress.

Because treatment first requires recognition of the condition, methods have been developed to identify when an individual is suffering from stress. These methods generally involve assessing certain stress indicators such as heart rate, respiration, and skin conductivity at one point in time. Although such measurements may be indicative of acute stress, in order to recognize chronic stress, there is a need for a method of measuring stress over an increased period of time. Although physical measurements such as electrocardiograms, arterial pressure, respiratory volume, and integrated nerve activity may be impacted by chronic stress, at any one time a single measurement is not conclusive of chronic stress. There is a need for monitoring stress levels and associated metrics over an increased period of time because while chronic stress and its effects are acknowledged, the impact of the chronic stress may go unnoticed. For example, blood pressure may gradually increase over time and may not cause any noticeable symptoms until one suffers extensive damage.

Furthermore, determining the effectiveness of a treatment option requires an analysis of certain physiological data over an increased period of time. In order to determine whether a treatment has been effective, or what treatments are more effective than others, quantifiable physiologic metrics must be monitored over time and presented such that an individual can assess how treatment options or lifestyle changes have positively, or negatively, impacted their stress level and associated health.

Accordingly, there is a need for a stress monitoring system and method capable of providing information regarding a body's response to stress over an increased period of time in order to more effectively monitor change in stress level thereby aiding in the management and treatment of stress.

### BRIEF DESCRIPTION OF THE DRAWINGS

These and other features and advantages will be better understood by reading the following detailed description, taken together with the drawings wherein:

FIG. 1 is a high level block diagram of one embodiment of a stress level monitor system configured to acquire and provide physiologic data.

FIG. 2 is a flowchart of an exemplary software program configured to receive and provide physiologic data.

FIG. 3 is a screenshot of an exemplary calibration window.

FIG. 4 is a screenshot of an exemplary time series window.

FIG. 5 is a screenshot of an exemplary analysis window.

FIG. 6 is a screenshot of an exemplary correction window.

FIG. 7 is an exemplary questionnaire for the input of psychological data.

### DETAILED DESCRIPTION

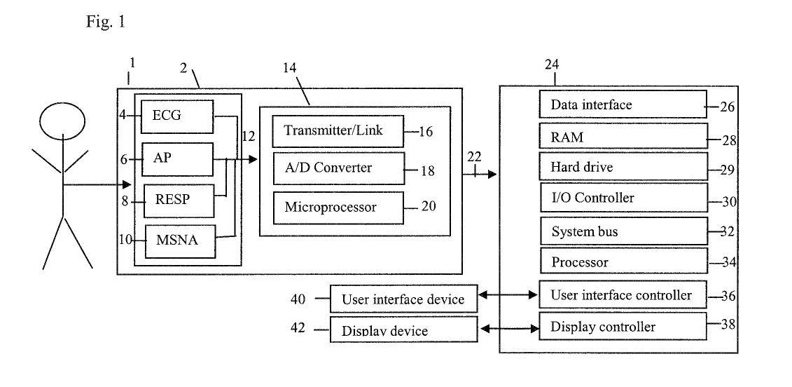

Referring to FIG. 1, one embodiment of a stress monitor system is shown generally as including a physiologic data transmitter 2 and a physiologic data receiver 24. The physiologic data transmitter 2 can include physiologic sensors 1 and a physiologic data processor 14. The physiologic data processor 14 can also be attached to the physiologic data receiver 24. In the preferred embodiment, the physiologic data sensors 1 can include an electrocardiogram (ECG) sensor 4, an arterial pressure (AP) sensor 6, a respiratory volume (RESP) sensor 8, and a muscle sympathetic nerve activity (MSNA) sensor 10, for example. The ECG sensor 4 can be an electrocardiograph having electrodes selectively placed on the body, the AP sensor 6 can be a blood pressure meter such as a sphygmomanometer, the RESP sensor 8 can be a pulmonary function test including a spirometer configured to output a signal proportional to airflow, and the MSNA sensor 10 can include microelectrode recordings of muscle sympathetic nerve activity from the peroneal nerve in the leg, for example, or any other type of sensor, or combination of sensors, as is well known in the art.

MSNA is a measure of the sympathetic nervous system and thus indicates the stress a person may be experiencing at any given time. ECG, AP, and RESP are measurements of the physical condition of the heart, lungs, circulatory, and respiratory systems. Stress has an adverse impact on these organs and systems and, therefore, the MSNA, ECG, AP, and RESP physiologic input parameters, considered together or separately, can be effective indicators of stress level and stress experience when monitored and analyzed over a period of time. However, other input parameters such as galvanic skin response and body temperature, for example, are contemplated as relevant and effective indicators of stress level and can be used as input parameters in place of, or in conjunction with, the input parameters of the preferred embodiment as discussed above.

One or more of the physiologic sensors 1 can have an analog output. Raw analog physiologic data 12 can be sent to an analog to digital converter 18 in the physiologic data processor 14 where the data 12 can be converted into digital format if necessary. In the embodiment shown in FIG. 1, once converted into digital format, a microprocessor 20 can control a transmitter/link 16 which is configured to send the raw digital physiologic data 22 to the data interface 26 of the physiologic data receiver 24. The transmitter/link 16 and data interface 26 can be physically connected by using a universal serial bus (USB) interface or wirelessly connected by using a IEEE 802.115 Bluetooth or IEEE 802.11 Wi-Fi interface for example. In another embodiment, physiologic data processor 14 is included in the physiologic data receiver 24 and, therefore, no transmitter/link 16 is required.

Still referring to FIG. 1, the physiologic data receiver 24 can have volatile memory such as random access memory (RAM) 28, non-volatile memory such as a conventional hard drive 29, a processor 34, a system bus 32 configured to move information among receiver 24 devices, an input/output controller 30 configured to connect peripheral devices such as a disk drive, a user interface controller 38 configured to receive and send signals from a user interface device 40 such as a conventional mouse, keyboard, or trackball, a display controller 38 configured to send and receive signals from a display device 42 such as a conventional monitor or screen, and a data interface 26 as discussed above. The data receiver 24 can also be a personal digital assistance (PDA), a conventional personal computer (PC), a smartphone, or any other computing device, for example.

To acquire raw digital physiologic data 22, a person interacts with the physiological sensors 1 to produce signals which can be processed by a physiologic data processor 14 and, if in analog format, converted into digital format, and sent, directly or wirelessly, if necessary, to a physiologic data receiver 24. The raw digital physiologic data 22 can then be stored in a database on a hard drive 29. The raw digital physiologic data 22 can then be accessed by a computer software program, for example a Windows®-based C++ program stored on a hard drive 39 or a compact disc, for example, which can access raw digital physiologic data 22 from a database in which the data 22 can be stored.

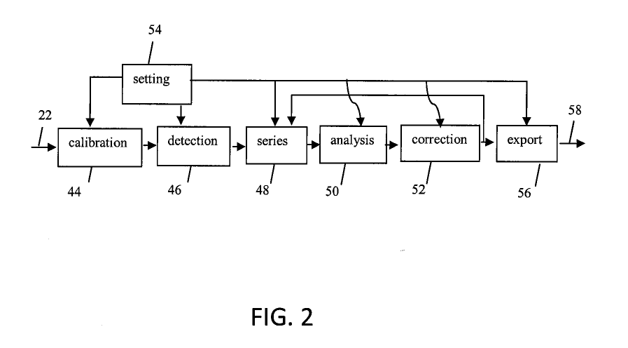

FIG. 2 shows a flowchart of a software-implemented method of monitoring stress including receiving raw digital physiologic data 22 stored on a hard drive 29. As described in more detail below, this raw digital physiologic data 22 can be interpreted in a calibration step 44 capable of extracting meaningful information from raw analog to digital conversion values. After calibration, the step of detection can include running detection routines configured to extract physiologic data from the physiologic sensors 1 that is now meaningfully interpreted by the software program and capable of being added to the user's profile as described in more detail below. The next step can be a series step 48 wherein the physiologic data can be displayed in an interactive window such that the data, along with limits and boundaries for example, can be viewed and manipulated by a user. The next step as shown in FIG. 2 and described further below, can be an analysis step including performing various meaningful calculations on the data to allow for more effective interpretation of the data. The next step can be a correction step wherein the data is corrected to account for missed, under, or over detections as well as interpolated so as to smooth the graphical representation of the data. At this point the user can return the corrected data to a series step 48 where the series, analysis, and correction steps can be repeated or the data can be exported in an export step to a text file or to an Interbase/Firebird database, for example, where it can be stored. This stored data can represent a user profile as described further below. It should be noted that before, during, or after any step, the step of setting 54 can be performed allowing a user to customize the program output, access tutorials which guide the user over a complete analysis and explain how to interpret the extracted data, or edit the parameters used for the analysis procedures, for example.

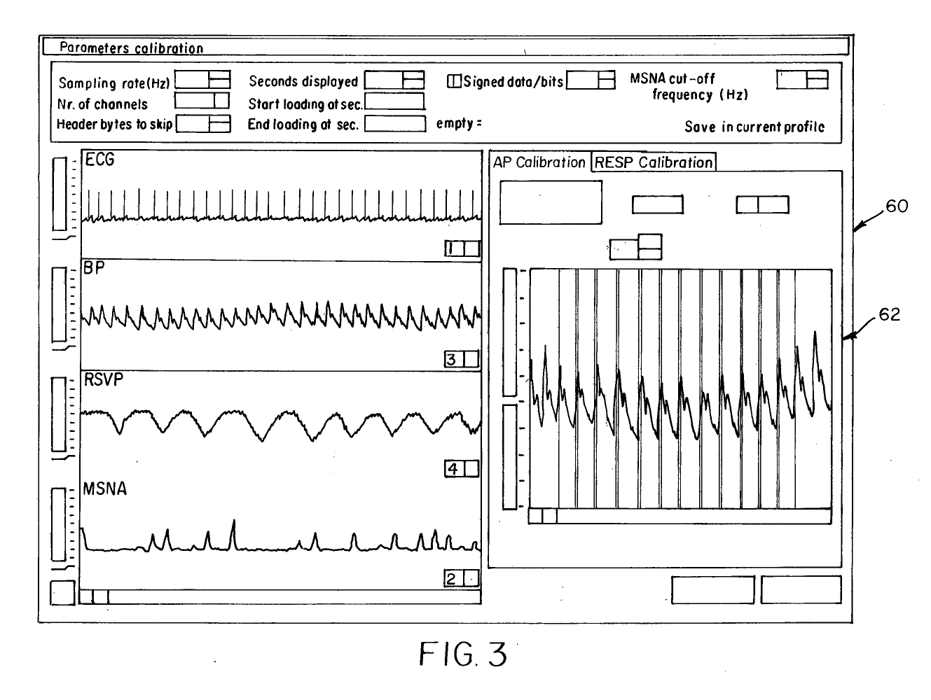

Referring now specifically to FIG. 3, a screenshot of an exemplary calibration window 60 is shown as one embodiment of calibration step 44. Input raw digital physiologic data 22 may not be calibrated. For example, the sample representation shown in FIG. 3 includes data expressed in quanta values received from an analog to digital conversion. In the example shown in FIG. 3, a calibration is necessary to extract meaningful AP and RESP values. FIG. 3 shows the maximum and minimum AP calibration window 62 where a user can calibrate the signal either acting on a single-wave, associating maximum/minimum AP values to a single selected peak/valley pair, or on multiple waves, assigning minimum/maximum AP values to the maximum/minimum averages computed over several peak/valley pairs. In the embodiment shown in FIG. 3, the RESP signal can undergo similar single-wave, maximum and minimum calibration. The calibration window 60 can also allow for visualization of each of the signals as indicated, for example, by the four graphical representations on the left side of FIG. 3.

After calibrating, a user can cause the software program to run detection routines so as to evaluate the signals, after conversion into digital form, sent from the physiological sensors 1. The digital data and evaluations can include ECG, heart period (HP) measured as the temporal distance between two successive QRS complexes, systolic AP (SAP) measured as the AP maximum in the current HP, diastolic AP (DAP) measured as the AP minimum after the current SAP, mean MSNA in the current HP, MSNA bursts including their rate, amplitude and area, and RESP volume measured once per cardiac beat at the beginning of the current HP, among others.

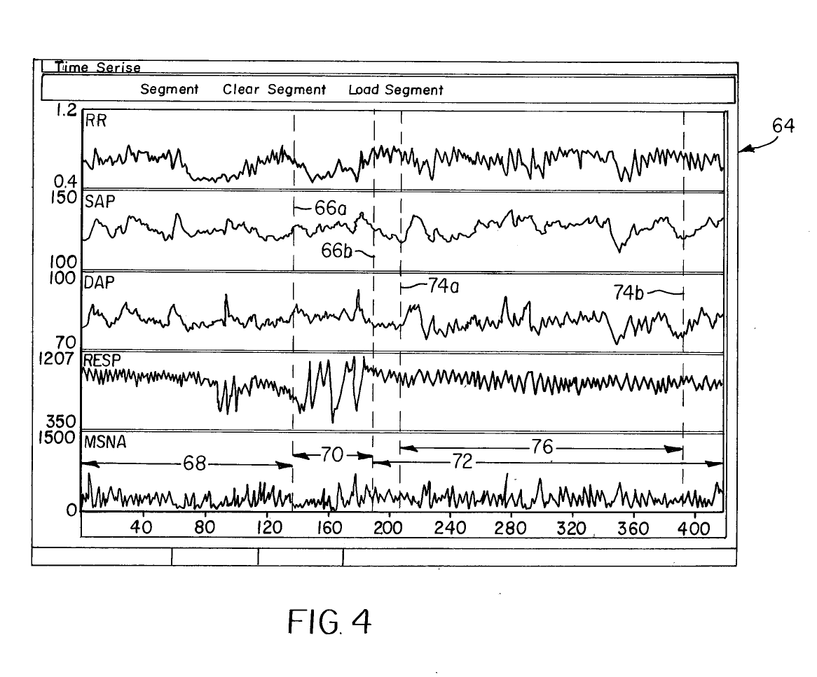

Referring now to FIG. 4, a screenshot of an exemplary time series window 64 is shown as one embodiment of series step 48. Once the physiologic digital data has been detected in step 46, it can be displayed to a user in series step 48 such that the user can manipulate the data. For example, a user can rescale each series by engaging the user interface device, such as a conventional computer mouse, at any point on the y-axis. A user can also control segment boundaries 66 which can be inserted or deleted by clicking the right mouse button, for example, on the graph. Segment boundaries 66a, 66b can be used to designate the start and the end of multiple sessions during the same reading. A user can also choose a reference segment which can be used to normalize indexes derived from other segments, enable analysis of a segment, or disable analysis of a segment by clicking on the right mouse button and engaging a popup menu, for example. For example, in FIG. 4, a reference segment 68, a disabled segment 70, and an enabled segment 72 are shown. Different background colors can also be used to help visualize special meaning associated with each segment, such as its status as reference, enabled or disabled, for example. A user can also engage a user interface device 40 to set analysis limits 74a, 74b that are different from segment boundaries as indicated by grey portion 76 of enabled segment 72 in FIG. 4, for example.

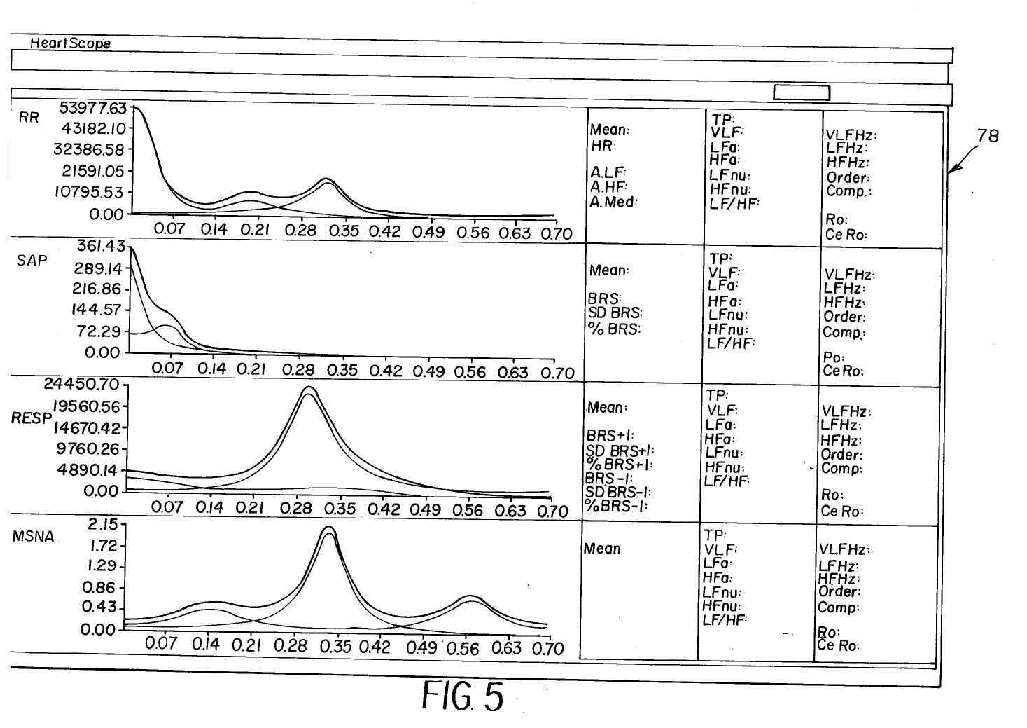

Referring now to FIG. 5, a screenshot of an exemplary analysis window 78 is shown as one embodiment of analysis step 50. Once a user has manipulated the physiologic data, the data can be analyzed according to the analysis limits indicated by the user in step 48 such as the analysis limits 74a, 74b shown in FIG. 4. The software program can calculate mean and variance, mean burst rate, burst amplitude and area normalized with respect to those calculated in the reference segment 68, for example, autoregressive (AR) power spectra and powers in the low and high frequency (LF and HF) bands, bivariate AR phase spectra and squared coherence between all pairs of series as a function of the frequency and at specific reference frequencies in LF and HF bands, the baroreflex gain, the magnitude of the HP-SAP transfer function, the slope of the response of the HP-SAP block to a simulated unitary ramp after the identification of an exogenous (X) model with an AR input (XAR model) or of a double X model with an AR input (XXAR model), the gain of the SAP-RESP and HP-RESP transfer functions in the HF band, indexes of complexity; a parameter related to the dynamical properties of the sinus node, and parameters from symbolic analysis quantifying the rate of occurrence of patterns lasting three cardiac cycles, for example and as such calculations are well known in the art. In addition to the calculations on the input parameters noted above, further physiologic output parameters, as shown in Table 1 below, can be instructive with respect to analyzing the input parameters and the individual's stress level.

TABLE 1
Output Paramters

| Output Paramater | Description |
|---|---|
| DAP | Mean of the diastolic arterial pressure values of arterial pressure signal |
| RR | Mean of the time intervals from an R peak to the subsequent one on the ECG |
| SAP | Mean of the systolic arterial pressure values on arterial pressure |
| Ro.RR | Regularity index measuring the normalized amount of information carried by |
| | the RR series |
| Ce.RR | Minimum of the corrected conditional entropy measuring the amount of |
| | information carried by the RR series |
| TP.RR | Variance of the RR series |
| TP.SAP | Variance of the SAP series |
| HFa.RR | Absolute spectral power in the High Frequency band calculated over the RR |
| | series |
| LFnu.RR | Absolute spectral power in the Low Frequency band divided by variance |
| | minus the power in the Very Low Frequency band calculated over the RR |
| | series |
| LF/HF.RR | Ratio of the absolute spectral power in the Low Frequency band and the |
| | absolute spectral power in the High Frequency band calculated over the RR |
| | series |
| LFa.SAP | Absolute spectral power in the Low Frequency band calculated over the SAP |
| | series |
| A.LF | Baroreflex gain in Low Frequency band (the square root of the ratio between |
| | LF spectral powers of RR and SAP). |
| A.HF | Baroreflex gain in High Frequency band (the square root of the ratio between |
| | HF spectral powers of RR and SAP). |
| A.Med | Average value between A.LF and A.HF |
| BRS | Baroreflex gain calculated with the sequence method |
| A.XAR | Baroreflex gain calculated with an open loop exogenous autoregressive model |
| A.XXAR | Baroreflex gain calculated with an open loop double exogenous |
| | autoregressive model |
| %0v.RR | Percentage of patterns lasting 3 cardiac cycles (four beats) with no variation |
| | (all the symbols are equal) calculated over the RR series |
| %1v.RR | Percentage of patterns lasting 3 cardiac cycles (four beats) with one variation |
| | (two consecutive symbols are equal and the remaining one is different) |
| | calculated over the RR series |
| %21v.RR | Percentage of patterns lasting 3 cardiac cycles (four beats) with two like |
| | variations (the three symbols form an ascending or descending ramp) |
| | calculated over the RR series |
| %2uv.RR | Percentage of patterns lasting 3 cardiac cycles (four beats) with two unlike |
| | variations (the three symbols form a peak or a valley) calculated over the RR series |

Although the calculation of the output parameters has been described as performed in analysis step 50, it can also be performed on the input data prior to manipulation in step 48 or after correction of the input data at step 52 and as described below.

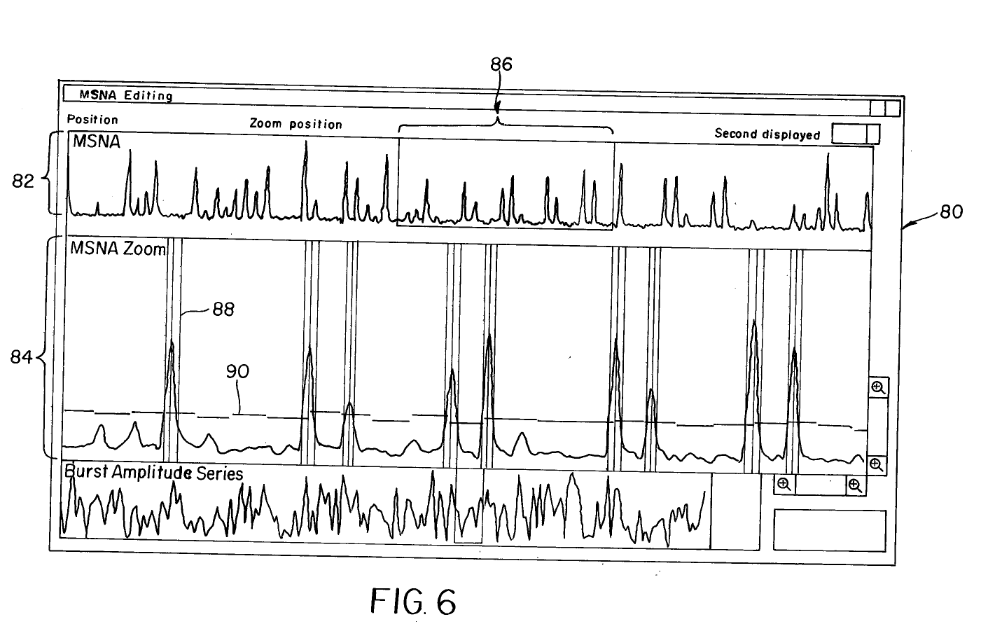

Referring now to FIG. 6, a screenshot of an exemplary correction window 80 is shown as one embodiment of correction step 52. After the input data has been analyzed in analysis step 50, the input data can be corrected. Correction window 80 shows an example of an MSNA correction window but other correction windows can include HP correction windows and SAP correction windows, among others. An HP correction window can allow for the insertion of under detections and the removal of over detections and both HP correction and SAP correction windows can allow for cubic spline interpolation over consecutive values to smooth successive outliers. The MSNA correction window 80 shows MSNA depicted over two different time scales (upper 82 and middle 84 panels). The upper panel 82 can allow for scrolling of the signal. The middle panel 84 can display the selected portion 86 of the MSNA signal from the upper panel 84. Onsets, peaks, and offsets of the detected bursts are marked with vertical segments such as vertical segments 88 for example, while the horizontal lines such as horizontal line 90 for example, can indicate the running threshold which can be updated on a beat-to-beat basis. The manual insertion or cancellation of any detection can be carried out by engaging a user interface device 40 such as by clicking a right mouse button on the middle panel 84.

Referring to FIG. 2 the step of exporting 56 can be performed by a user in conjunction with the software program once the user decides to create or update a user profile 58 with data from the current reading. A user profile 58 is a text file or database table(s) or entry(ies) that can include historical data from a plurality of readings so as to provide a user with the ability to compare current readings and prior readings.

In particular, one or more physiologic data parameters based on historical data can be compared to one or more current readings of that, or any other, physiologic data parameter. The historical data can include an average of all prior readings calculated as (ER)/N, for example, where R represents each reading of a given physiologic data parameter and N is the total number of such readings for that physiologic data parameter. The historical data can also be mined to show other relevant indicia, such as a running average of the most recent number of readings n, were n is any integer equal to or less than N. The average, derived from the normalized, historical experience of the patient, can provide an individual or medical professional with an indication of how much the current readings vary from the normal or average readings for that specific individual. Therefore, an individual or medical professional can monitor and assess the individual's level of stress over an increased period of time in order to determine the presence of chronic stress and/or monitor and assess the effect of a treatment option(s) on chronic stress. Accordingly, the software program can be configured to retrieve stored historical data from a text file or database and calculate the relevant average(s) for the relevant parameter(s), as the average, parameter, and time range(s) are specified by a user's input. The software program can also be configured to display the specified calculations along with one specific reading, such as the most recent reading for example, or a range of readings having time limits less than those time limits used in the calculations, such as the most recent week or month if the time limit used in the calculations was past year for example.

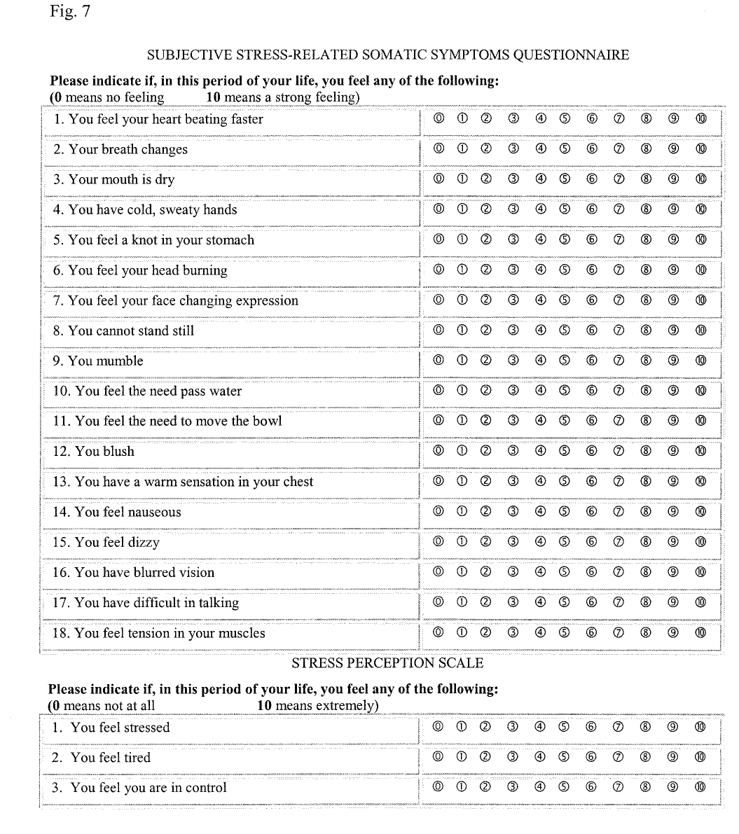

Although the invention thus far has been described as including the acquisition of physiological, quantitative data, the assessment of an individual's stress, including changes over time and the success of treatment methods, can be better understood by viewing the physiologic input parameters in combination with psychological, more qualitative data. FIG. 7 shows an example of an optional questionnaire including questions relating to somatic and stress perception questions. The questions listed in FIG. 7 are exemplary only and any number of questions can be asked of an individual in order to assist in stress assessment and monitoring.

Accordingly, at any point in the process shown in FIG. 2, but preferably before calibration 44 and/or export 56, the software program can cause a questionnaire to be displayed, such as that shown in FIG. 7, for example, and the user can subjectively answer the questions shown by interacting with the software through a user interface device. The user's answers can then be exported by the software program similar to the export step 56 for the physiologic data. Accordingly, the user's answers can be stored in the user profile 58 which can include historical data from a plurality of questionnaire answers so as to provide a user with the ability to compare current answers with prior answers as well as the change and average answer over time to a specific question(s). Since the user profile 58 is also configured to store physiologic data, the software program can be configured to display, preferably in a graphical format, both historical physiologic and psychological data to allow for a better understanding and assessment of an individual's stress over time.

It should be noted that while the comparison over time of physiologic and psychological data has been described as using only data acquired by one individual, in another embodiment, an individual's physiologic and psychological data can be analyzed more objectively using the data acquired from other individuals and, preferably, the average of such data. Accordingly, an objective standard can be computed by the software, which preferably stores user profiles 58 in a database, and the software can optionally be configured to allow a user to access the database to retrieve at least a portion of another user's data primarily for comparison purposes and/or for the purpose of average calculation. Accordingly, the acquired data can be limited by factors such as age, weight, or psychological data such as those individuals who have a strong feeling of blurred vision or cold, sweaty hands, for example.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. A stress monitoring method, comprising the steps of:
acquiring a plurality of individual readings of at least one physiologic data parameter over a period of time;
storing the plurality of individual readings;
determining the average of at least a portion of the plurality of individual readings; and
comparing at least one individual reading to the average to identify any differences between the average and the at least one individual reading.

2. The stress monitoring method of claim 1 wherein the at least one physiologic data parameter is selected from the group consisting of electrocardiograms (ECG), arterial pressure (AP), respiratory pressure (RESP), integrated nerve activity (MSNA), galvanic skin response, and body temperature.

3. The stress monitoring method of claim 1 wherein the step of storing the plurality of individual readings includes storing the plurality of individual readings in a storage device, the storage device being selected from the group consisting of a text file and a database.

4. The stress monitoring method of claim 1 wherein the step of determining the average further includes receiving input from a user, the input from a user including begin date, end date, and physiologic data parameter(s), calculating the total number of individual readings occurring between the begin date and the end date, inclusive, calculating the sum of each of the selected physiologic data parameter(s) for the period from the begin date to the end date, inclusive, and dividing the sum of each of the selected physiologic data parameter(s) by the total number of individual readings.

5. The stress monitoring method of claim 1 wherein the step of determining the average further includes receiving input from a user, the input from a user including number of previous readings and physiologic data parameter(s), calculating the sum of each of the selected physiologic data parameter(s) for the number of previous readings selected by the user, and dividing the sum of each of the selected physiologic data parameter(s) by the number of previous readings.

6. The stress monitoring method of claim 1 wherein the step of acquiring a plurality of individual readings further includes acquiring at least one psychological data parameter over a period of time.

7. A computer program product embodied in a computer readable medium for stress monitoring comprising programming instructions for:
acquiring at least one individual reading of at least one physiologic data parameter over a period of time;
appending a user profile to include the at least one individual reading;
determining the average of a plurality of individual readings of the user profile; and
displaying a comparison of at least one individual reading to the average.

8. The computer program product of claim 7 wherein the at least one physiologic data parameter is selected from the group consisting of electrocardiograms (ECG), arterial pressure (AP), respiratory pressure (RESP), integrated nerve activity (MSNA), galvanic skin response, and body temperature.

9. The computer program product of claim 7 wherein the user profile is a storage device, the storage device being selected from the group consisting of a text file and a database.

10. The computer program product of claim 7 wherein the programming instructions for determining the average further include receiving input from a user, the input from a user including begin date, end date and physiologic data parameter(s), calculating the total number of individual readings occurring between the begin date and the end date, inclusive, calculating the sum of each of the selected physiologic data parameter(s) for the period from the begin date to the end date, inclusive, and dividing the sum of each of the selected physiologic data parameter(s) by the total number of individual readings.

11. The computer program product of claim 7 wherein the programming instructions for determining the average further include receiving input from a user, the input from a user including number of previous readings and physiologic data parameter(s), calculating the sum of each of the selected physiologic data parameter(s) for the number of previous readings selected by the user, and dividing the sum of each of the selected physiologic data parameter(s) by the number of previous readings.

12. The computer program product of claim 7 wherein the programming instructions for acquiring the at least one individual reading further include calibrating at least one individual reading, detecting at least one individual reading, manipulating at least one individual reading according to input from a user, analyzing at least one individual reading, and correcting at least one individual reading.

13. The computer program product of claim 7 wherein the programming instructions further include settings configured to allow a user to customize the program output, access tutorials, and edit the parameters used.

14. The computer program product of claim 7 wherein the step of acquiring a plurality of individual readings further includes acquiring at least one psychological data parameter over a period of time.

15. The computer program product of claim 9 wherein the step of determining the average includes retrieving the user profile of at least one other individual from the storage device and determining the average of a plurality of individual readings of the at least one other individual.

16. A stress monitoring system, comprising:
at least one physiologic sensor configured to acquire physiologic data;
a physiologic data processor configured to transmit physiologic data acquired by the at least one physiologic sensor;
a physiologic data receiver configured to receive physiologic data from the physiologic data processor, the physiologic data receiver including circuitry operable to store physiologic data; and
a display device configured to receive physiologic data from the physiologic data receiver, the display device being configured to graphically display physiologic data to a user.

17. The stress monitoring system of claim 16 wherein the at least one physiologic sensor is selected from the group consisting of an electrocardiograms (ECG) sensor, arterial pressure (AP) sensor, respiratory pressure (RESP) sensor, integrated nerve activity (MSNA) sensor, galvanic skin response sensor, and body temperature sensor.

18. The stress monitoring system of claim 16 wherein the physiologic data processor includes an analog to digital converter configured to receive analog signals from the at least one physiologic sensor and convert analog signals into digital signals.

19. The stress monitoring system of claim 16 wherein the physiologic data processor includes a transmitter device wherein the transmitter device is selected from the group consisting of a universal serial bus interface, a Bluetooth interface, and a Wi-Fi interface.

20. The stress monitoring system of claim 16 wherein the physiologic data receiver includes a data interface device wherein the data interface device is selected from the group consisting of a universal serial bus interface, a Bluetooth interface, and a Wi-Fi interface.

21. The stress monitoring system of claim 16 wherein the circuitry operable to store physiologic data is selected from the group consisting of random access memory, a magnetic disk drive, and an optical disk drive.

22. The stress monitoring system of claim 16 wherein the display device is selected from the group consisting of a cathode ray tube, plasma, liquid crystal, thin-film transistor, light-emitting diode, and organic light-emitting diode.

23. The stress monitoring system of claim 16 wherein the physiologic data receiver further includes a processor, a system bus, an input/output controller, a user interface controller, a user interface device configured to engage the user interface controller, and a display controller configured to engaged the display device.

24. The stress monitoring system of claim 16 wherein the physiologic data receiver is a selected from the group consisting of a personal computer, a personal digital assistance, a cellular telephone, and a smartphone.
